(19) United States
(12) Patent Application Publication
(10) Pub. No.: US 2006/0254581 A1
(43) Pub. Date: Nov. 16, 2006

(54) Dose counting in metered dose inhaler

(76) Inventors: Perry Genova, Apex, NC (US); Dan Deaton, Apex, NC (US); Matt Khare, Willow Springs, NC (US)

Correspondence Address: KOS PHARMACEUTICALS, INC., 1 CEDAR BROOK DRIVE, CRANBURY, NJ, 08512-3618, US

(21) Appl. No.: 11/431,687

(22) Filed: May 9, 2006

Related U.S. Application Data
(60) Provisional application No. 60/594,852, filed on May 12, 2005.

(51) Int. Cl. A61M 11/00; G08B 3/00; A62B 27/00

(52) U.S. Cl. 128/200.23; 128/202.22

(57) ABSTRACT

A counter for indicating the number of doses left in a canister that is suitable for use in a metered dose inhaler. The counter is affixed to the canister and includes a module for providing an indication of the number of doses left in the canister and a triggering mechanism for updating the indication in response to activation of the inhaler.

### FIELD OF THE INVENTION

The present invention relates to electronic monitoring and counting of medication dosages, and in particular to a metered dose inhaler that includes an electronic counter module.

### BACKGROUND OF THE INVENTION

Metered dose inhalers (“MDI”) of various configurations are known for dispensing medication into the mouth or nasal passages of a patient. Medication is expelled from the actuator and inhaled by the patient and absorbed by the mouth, nose, throat and lungs. One example is the device commonly used by asthma sufferers for dispensation of airway opening drugs. These are often called “Press & Breathe” inhalers and require simple pressing on the canister and inhalation by the user.

A pressurized metered dose inhaler (“pMDI”) is designed to deliver therapeutic agents, e.g. medicaments, to the human respiratory tract or nasal cavity. Accordingly, the pMDI contains the active substance, dissolved or suspended, in a fluid propellant system that contains at least one liquefied gas in a pressurized container that is sealed with a metering valve. The actuation of the valve delivers a metered dose of medicament in the form of an aerosol spray and is directed by a suitable adapter/activator for dispensation via oral or nasal inhalation.

Another type of inhaler is the breath-activated inhaler (“BAI”). A BAI is a device typically for use with a pressurized metered dose inhaler system, and is comprised primarily of an inhalation sensing means, a means to actuate the canister automatically upon an appropriate inhalation profile, and a triggering means to communicate between the two. A BAI can be of any conventional design that has or is capable of being adapted to have, using any conventional means, such as mechanics, electro mechanics, pneumatics, fluid dynamics, a trigger pressure drop of about 0.1 to about 20 cm of water pressure. The “trigger force” refers to the force that is minimally required by the patient to activate the dosing mechanism associated with the device. The breath-activated inhaler typically uses the suction of the user as the triggering force to release the medication.

Inhalation may be sensed by measuring changes in pressure through the device or by measuring flow rate, directly or indirectly and separately or in combination. The literature is replete with methods for accomplishing this and includes moving vanes or flaps, elastomeric diaphragms, electronic pressure sensors, flow sensors, and combinations of mechanical sensors with electronic timing circuits.

The canister may be actuated by mechanical (e.g. springs, levers, etc.) electromechanical (e.g. solenoids, motors) or pneumatic means. The canister may be actuated and remain in the actuated position until intervened upon by the patient or may be caused to dwell in the actuated position for some duration returning automatically to rest position without any intervention.

Traditional MDI inhaler devices are known to be confusing to the user with respect to the number of medication containing actuations remaining in the canister at any one time. Accordingly, the user is faced with the possibility of running out of necessary medication at a critical time. Alternatively, the user must carry additional costly medication at all times to insure that it is always on hand. Further, the disposal of a canister of medication when there are still a number of doses remaining can lead to increased expense in the treatment of an ailment.

Still further complications with the traditional inhalers mean that a user is forced to manually determine the timing between dosing. As a result it is up to the user to insure that a proper time period has expired between dosing to prevent an overdosing of medication. Similarly, many medications have a maximum threshold for dosing over a specific period. As a result overdosing can occur when more than the predetermined number of actuations are administered in a set period, for example 24 hours. Once again it is up to the user to ensure that no more than the maximum number of actuations is administered over the time period. In addition, the medications may require a sequence of multiple device activations to deliver a complete dose. The user must accurately monitor these activations. With the state of current medical treatments, often a user will have multiple drugs prescribed for the treatment of a single malady. When coupled with the irregularity of the dosing schedules improper dosing of a patient becomes a genuine concern.

### SUMMARY OF THE INVENTION

In view of the drawbacks associated with prior inhalers, the inventors have recognized the desirability of tracking the use history of a reservoir device that is used for delivering drugs to the lungs of patients for the purpose of treating local and systemic diseases. Accordingly, the present invention is directed toward such tracking

In an illustrative embodiment, the invention is implemented in a dose counter for indicating the number of doses left in a canister that is suitable for use in a metered dose inhaler. The counter is affixed to the canister and includes a module for providing an indication of the number of doses left in the canister and a triggering mechanism for updating the indication in response to activation of the inhaler. As an optional feature, the counter can provide an indication of the number of doses taken as part of a predetermined dosage sequence, and/or the number of doses remaining to be taken as part of a predetermined dosage sequence.

### BRIEF DESCRIPTION OF THE DRAWINGS

The following detailed description, given by way of example and not intended to limit the present invention solely thereto, will best be appreciated in conjunction with the accompanying drawings, wherein like reference numerals denote like elements and parts, in which:

FIG. 1A shows a pressurized metered dose inhaler (pMDI) in accordance with the invention;

FIG. 1B shows the pMDI of FIG. 1A disassembled into a canister assembly and an actuator;

FIG. 2 is a plan view of a pMDI in accordance with a first embodiment of the invention;

FIG. 3 is a plan view of a pMDI in accordance with a second embodiment of the invention;

FIG. 4 is a plan view of a pMDI in accordance with a third embodiment of the invention;

FIG. 5 is a plan view of a pMDI in accordance with a fourth embodiment of the invention; and

FIG. 6 is a plan view of a pMDI in accordance with a fifth embodiment of the invention.

### DETAILED DESCRIPTION OF THE INVENTION

In view of the drawbacks associated with prior inhalers, the inventors have recognized the desirability of tracking the use history of a reservoir device, such as an inhaler, that is used for delivering drugs to the lungs of patients.

It should be noted that while the invention will be described in the context of dispensing a medicament from canister, it is possible to use the invention for dispensing materials other than medicament from the canister. For example, a breath freshener or candy may be dispensed. Further it will be appreciated by those of skill in the art that the administration of a dose may comprise a single actuation of the device, multiple actuations of the device in succession, or multiple single actuations over a period of time. Further, the term dose as used herein is interchangeable with sprays, puffs, or actuations, administrations, and other terms known to those of skill in the art to signify an available or administered medication release or amount, whether used singularly or to identify a plurality of such actions or amounts.

In one medical implementation of the invention, the invention provides means for indicating to a patient the number of puffs or actuations remaining in a pressurized metered dose inhaler (pMDI). An additional function provides for display of the number of metered puffs or actuations in a sequence when multiple actuations are required to achieve particular medication levels. The counter/display devices employed in the invention are affixed to the medicament canister. Counting function occurs when a switch coupled to the event counter circuit board is triggered in response to the displacement of the drug canister within an actuator body.

The display may carryout any number of functions including displaying the number of actuations remaining in the canister, the number of doses remaining in the canister, the time since the last administration, the time till the next administration, an alarm either visual, or audible, or both, administrations over a given time period, and warnings against over administration. Other display options are also available as would be understood by those of skill in the art.

In this description several alternative embodiments are presented for incorporating an Electronic Dose Counter (EDC) within a pMDI. In each embodiment the counter module is affixed to the pMDI canister, and is thus differentiated from an embodiment in which the counter is affixed or built into an actuator body. In the embodiments of the invention, the visual display is read from above the inhaler, or from a direction that is generally radial with respect to the major axis of the inhaler.

In each of the described embodiments, relative motion of the canister within the actuator body activates a switch component of the EDC module by means of a trigger mechanism positioned within a cap that is fixedly mounted to the canister. Thus, it is not the relative motion of the canister body or the valve which triggers the counting function, but the trigger mechanism mounted in the cap.

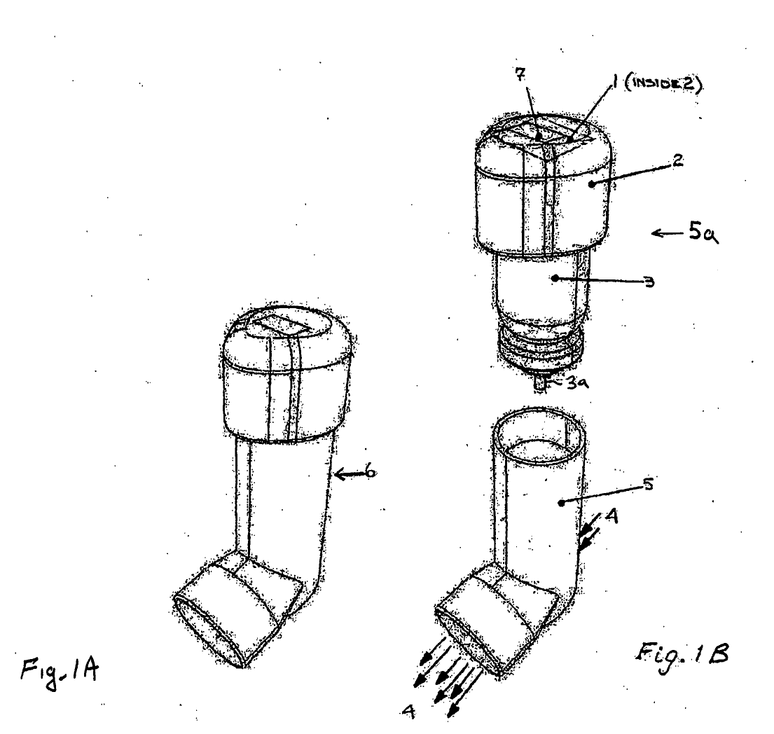

Preferably, the devices depicted in FIGS. 1A-6 are generally made of molded plastic, with the exception of the EDC module, the springs, seals and trigger elements. The springs are preferably made of metal and the seals and trigger elements are preferably made of elastomeric materials. Although, one skilled in the art of the invention will, upon review of this description, be able to substitute various materials for the preferable materials.

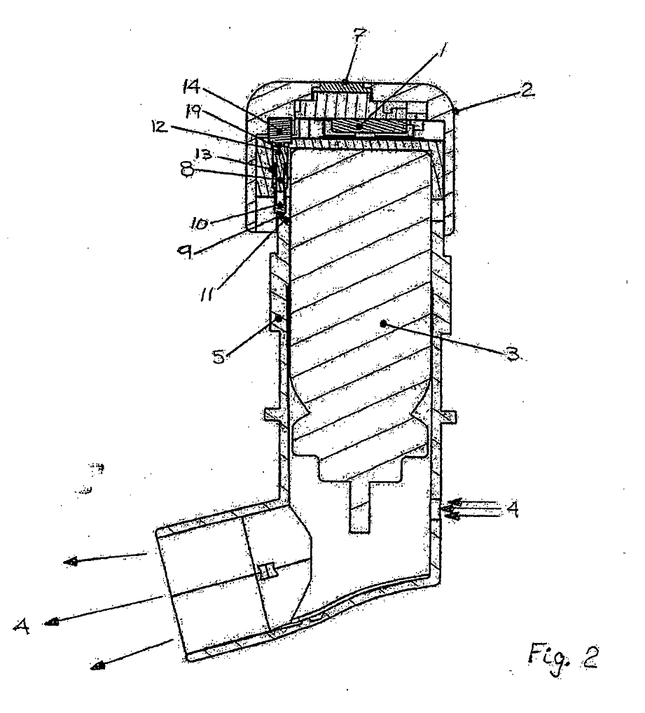

Referring now to FIGS. 1A and 1B, there is shown a pressurized metered dose inhaler (PMDI) 6 in accordance with the invention. As can be seen from FIGS. 1A and 1B, the inhaler is made up of an actuator 5 and a canister assembly 5a. The actuator is constructed to accommodate the canister assembly while providing an inhalation air pathway 4. The canister assembly includes a canister 3 for holding a medication or other material to be dispensed by the inhaler, an Electronic Dose Counter (EDC) circuit module 1 (as may be more clearly seen in FIG. 2), a Liquid Crystal Display (LCD) 7 for displaying dose count information to a user, and a cap 2 that houses the circuit module and liquid crystal display and is permanently secured to the canister upon manufacture.

The cap may be attached to the canister using various adhesives, through interference fit, or through snap fit. Regardless of methodology, the counter module 1 is isolated and sealed from the inhalation pathway 4 of the actuator body 5. This prevents any possible contaminants from entering the air pathway to or from the EDC module.

By fixedly attaching EDC 1 to canister 3 during manufacture, the numerical count displayed is always associated with the particular canister. When canister 3 is removed from actuator 5 for cleaning, the integral counter module in cap 2 comes with it as a unit. Thus, the counter and canister can not become separated, a condition potentially leading to an inaccurate count. Fully assembled, pMDI 6 is ready for use.

Upon actuation, the entire canister assembly is depressed and the cap 2 and canister 3 move together as a unit. At the appropriate point, just prior to the discharge of medicament via the metering valve, a switch on the EDC module is triggered as the cap moves toward the top edge of the actuator body 5 (see FIGS. 2-6). The design of such switching takes into account the appropriate travel distance of the canister to the drug discharge position with accommodation for over-travel to ensure that actuation of the EDC module never impedes the motion of the canister within the actuator body. At the end of travel of canister valve stem 3a, the assembly comes to a “hard stop.”

The EDC module is sealed in the cap as noted above. It is further noted that in some embodiments it may be desirable to key or index the canister assembly so that the assembly does not turn about the axis of the actuator. In other cases, the canister assembly and actuator may rotate independently of each other.

Referring now to FIG. 2, there is shown a plan view of a pMDI in accordance with a first embodiment of the invention. In the FIG. 2 embodiment, the LCD 7 is located on top of EDC module 1, and a trigger assembly 8 is mounted at a peripheral edge of cap 2. The trigger assembly is made up of a first plunger 10 in communication with an upper edge 11 of actuator body 5. A second plunger 12 is positioned about the proximal end of a spring 13 that is fitted between the first and second plungers. The second plunger is positioned to contact button 19 of switch 14 on EDC 1 circuit board. As a user urges the canister assembly down into the actuator body, upper edge 11 of the actuator body forces plunger 10 to compress spring 13 which, in turn, causes plunger 12 to contact button 19 to close switch 14. The closing of switch 14 causes the dose count to be updated. The updated dose count is displayed on LCD 7. Thus, update of the display count occurs at a predetermined point in the travel of canister 3 within actuator 5, and spring 13 absorbs any over travel of canister 3 after the discharge of medicament.

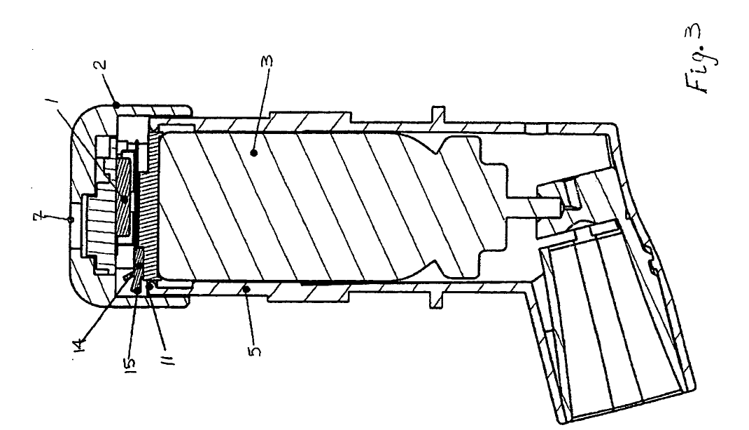

FIG. 3 is a plan view of a pMDI in accordance with a second embodiment of the invention. As in the FIG. 2 embodiment, the LCD is positioned at the top of the cap. The attachment of the cap to the canister is also the same as described in connection with the FIG. 2 embodiment. However, the switching mechanism in FIG. 3 differs from that of FIG. 2. In FIG. 3, count actuation is achieved through a horizontally mounted flip-up style switch 14 (e.g. PANASONIC P11152STR). When the canister is depressed relative to the actuator, switch 14 is closed to register a count, as upper edge 11 of the actuator deflects an integral, spring-biased, position-sensing arm 15 on switch 14. Over travel is accommodated within the switch mechanism itself.

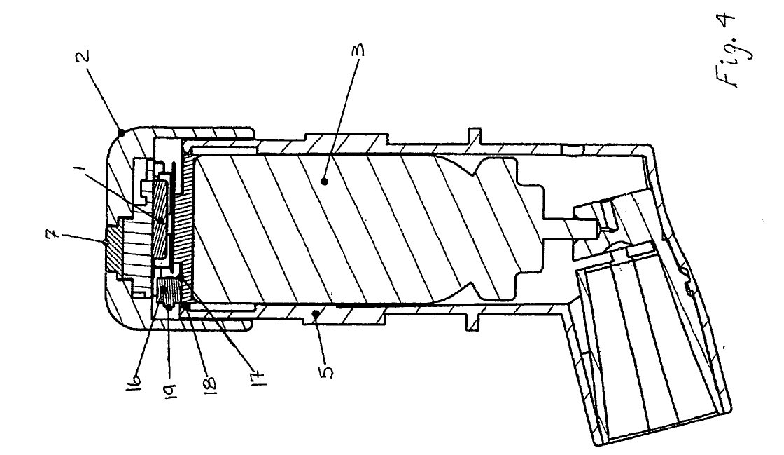

FIG. 4 is a plan view of a pMDI in accordance with a third embodiment of the invention. As in the FIG. 2 and FIG. 3 embodiments, the LCD is positioned at the top of the cap. Also the attachment of the cap to the canister is the same as described in connection with the FIGS. 2 and 3 embodiments. However, in the FIG. 4 embodiment, switching is achieved through a switch 16 that is mounted at an edge 17 of an EDC 1 circuit board. Orientation is such that the direction of actuation of switch 16 is parallel to the plane of the circuit board of EDC 1. A boss, or rib, 18 on the inside of upper edge 11 of actuator 5 slides against switch 16 depressing contact button 19 to initiate a count. Actuator 5 with rib 18 can then travel past the closure position of switch 16 by an amount sufficient to accommodate over travel without causing a false count by again actuating switch 16 on return travel.

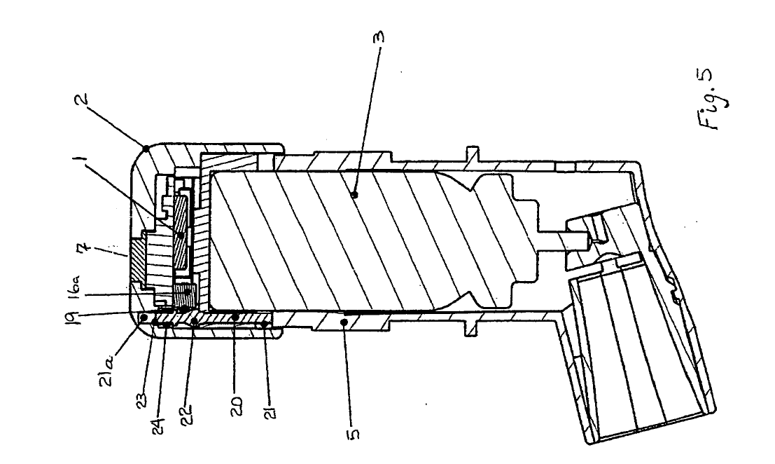

FIG. 5 is a plan view of a pMDI in accordance with a fourth embodiment of the invention. As in the FIGS. 2, 3 and 4 embodiments, LCD 7 is mounted at the top of the cap. A switch 16a is mounted to the EDC 1 circuit board and has a line of action that is horizontal relative to orientation of the depicted inhaler. The actuation mechanism is similar to the plunger mechanism in the device of FIG. 2. A plunger 20 is slideably retained in a cylindrical compartment 21 within the cap. Plunger 20 has a circumferential protrusion, or lobe, 22 near proximal end 23. A spring 24 is retained in a compartment 21a and engages the end of plunger 20. The distal end of plunger 20 rides on the upper edge 11 of actuator 5. Downward motion of the canister assembly relative to the actuator causes plunger 20 to move upward against the force of spring 24, raising lobe 22 and displacing contact button 19 in transit, thereby initiating a count. Over travel is accommodated by additional compression of spring 24. Range of motion is constrained to prevent an additional count upon return of plunger 20 to the “at rest” position. The FIG. 5 embodiment has potential advantages for sealing during manufacture.

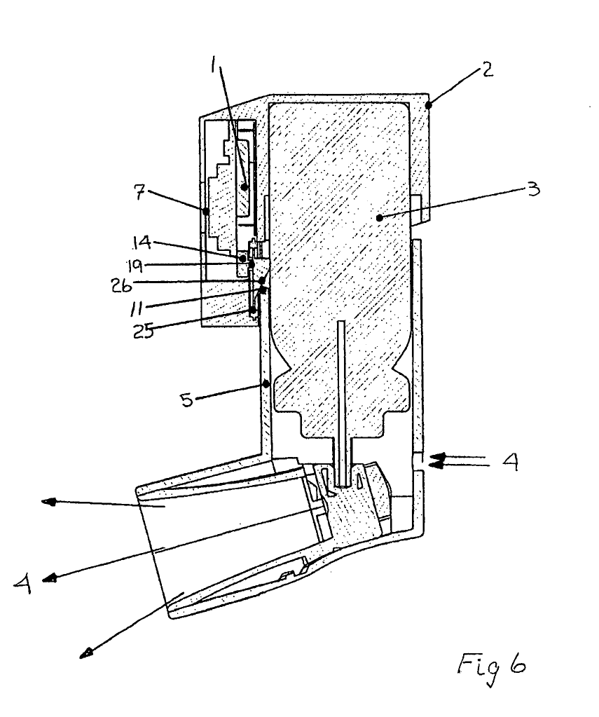

FIG. 6 is a plan view of a pMDI in accordance with a fifth embodiment of the invention. In the FIG. 6 embodiment, LCD 7 mounted on EDC 1 within cap 2 is attached to canister 3 such that it faces out from the side of the inhaler rather than facing up from the top of the inhaler. In this manner, the LCD 7 can be keyed to face the user when the user holds the inhaler in position to deliver medicament. The cap of the FIG. 6 embodiment is keyed to prevent rotation of the canister within the actuator body, thereby maintaining the appropriate orientation of the display. A switch 14 is mounted on EDC 1 circuit board such that contact button 19 is lodged against an elastomeric seal 25. A ramp shaped projection 26 is molded into seal 25 and is displaced by the upper edge 11 of actuator 5 as the canister assembly moves downward relative to the actuator body. The motion of the seal 25 and projection 26 against button 19 effects a display count on EDC 1.

Modifications to the present invention would be obvious to those of ordinary skill in the art in view of this disclosure, but would not bring the invention so modified beyond the scope of the appended claims.

What is claimed is:

1. A counter for indicating the number of doses left in a canister that is suitable for use in a metered dose inhaler, comprising:
a module for providing an indication of the number of doses left in the canister; and
a triggering mechanism for updating the indication in response to activation of the inhaler;
whereby the counter is affixed to the canister.

2. A counter as recited in claim 1, wherein the module is an electronic module.

3. A counter as recited in claim 1, wherein the triggering mechanism comprises a switch that is actuated in response to motion of the canister.

4. A counter as recited in claim 3, wherein the triggering mechanism further comprises at least one plunger for actuating the switch in response to motion of the canister.

5. A counter as recited in claim 3, wherein the triggering mechanism further comprises at least one plunger and a spring for actuating the switch in response to motion of the canister.

6. A counter as recited in claim 5, wherein the spring accommodates over-travel of the plunger.

7. A counter as recited in claim 2, wherein the switch is a flip-up style switch.

8. A counter as recited in claim 2, wherein the triggering mechanism further comprises an elastomeric member for actuating the switch.

9. A counter as recited in claim 1, wherein the module comprises a liquid crystal display.

10. A counter as recited in claim 1, wherein the module is operable to provide a count of the number of doses left in a predetermined sequence.

11. A counter as recited in claim 1, wherein the module can display one or more of the features selected from the group consisting of number of actuations remaining in the canister, the number of doses remaining in the canister, the time since the last administration, the time till the next administration, a visual alarm, an audible alarm, administrations over a given time period, and warnings against over administration.

12. A metered dose inhaler, comprising:
a canister assembly including a canister and a cap that is affixed to the canister and houses a counter for indicating the number of doses left in the canister; and
an actuator body for accommodating the canister assembly and for providing an inhalation air pathway.

13. A metered dose inhaler as recited in claim 12, wherein the counter comprises:
a module for providing an indication of the number of doses left in the canister; and
a triggering mechanism for updating the indication in response to activation of the inhaler.

14. A metered dose inhaler as recited in claim 13, wherein the module is an electronic module.

15. A metered dose inhaler as recited in claim 13, wherein the triggering mechanism comprises a switch that is actuated in response to motion of the canister assembly relative to the actuator body.

16. A metered dose inhaler as recited in claim 15, wherein the triggering mechanism further comprises at least one plunger for actuating the switch in response to motion of the canister assembly relative to the actuator body.

17. A metered dose inhaler as recited in claim 15, wherein the triggering mechanism further comprises at least one plunger and a spring for actuating the switch in response to motion of the canister assembly relative to the actuator body.

18. A metered dose inhaler as recited in claim 17, wherein the spring accommodates over travel of the plunger.

19. A metered dose inhaler as recited in claim 15, wherein the switch is a flip-up style switch.

20. A metered dose inhaler as recited in claim 15, wherein the triggering mechanism further comprises an elastomeric member for actuating the switch.

21. A metered dose inhaler as recited in claim 13, wherein the module comprises a liquid crystal display.

22. A metered dose inhaler as recited in claim 13, wherein the module is operable to provide a count of the number of doses left in a predetermined sequence.

23. A metered dose inhaler as recited in claim 12, wherein the counter can display one or more of the features selected from the group consisting of number of actuations remaining in the canister, the number of doses remaining in the canister, the time since the last administration, the time till the next administration, a visual alarm, an audible alarm, administrations over a given time period, and warnings against over administration.

24. A method of over-travel management in a metered dose inhaler comprising:
providing a counter for indicating the number of doses left in a canister that is suitable for use in the metered dose inhaler, a triggering mechanism for updating the counter in response to activation of the inhaler, the triggering mechanism further comprising at least one plunger and a spring for actuating the switch in response to motion of the canister;
depressing the canister; and
actuating the triggering mechanism wherein the spring accommodates over-travel of the plunger.
